(19) United States
(12) Patent Application Publication
(10) Pub. No.: US 2019/0099639 A1
(43) Pub. Date: Apr. 4, 2019

(54) GAIT TRAINING APPARATUS AND METHOD FOR PREVENTING OR LIMITING INJURIES

(71) Applicant: SUREFOOTED LLC, HOMER GLEN, IL (US)

(72) Inventors: Udai Jayakumar, Redondo Beach, CA (US); Jay Joshi, Woodridge, IL (US); Bradford Taras, Homer Glen, IL (US)

(21) Appl. No.: 16/208,025

(22) Filed: Dec. 3, 2018

Related U.S. Application Data
(63) Continuation of application No. 15/017,911, filed on Feb. 8, 2016, now abandoned.
(60) Provisional application No. 62/113,817, filed on Feb. 9, 2015.

(51) Int. Cl. A63B 24/00; A63B 69/00; A63B 23/04; A61B 5/11; A63B 22/20; A63B 22/02; A63B 21/00; A63B 71/00; A61B 5/00; A63B 21/005; G05B 19/05; A63B 26/00; A63B 21/008

(52) U.S. Cl. CPC A63B 24/0075; A63B 2220/22; A63B 23/0405; A61B 5/112; A63B 24/0087; A63B 24/0062; A63B 22/20; A63B 22/02; A63B 21/4041; A63B 21/4033; A63B 71/0054; A61B 5/7275; A61B 5/1117; G06F 19/00; A63B 21/00181; A63B 2220/52; A63B 2220/833; A63B 21/4007; A63B 2220/805; A63B 2220/72; A63B 2220/56; A63B 21/0058; A63B 2220/20; A63B 2024/0093; G05B 19/05; A63B 26/003; A63B 22/0235; A63B 21/0087; A63B 69/0064

(57) ABSTRACT

An apparatus and method for gait training is disclosed. The apparatus includes a support structure having a base and at least one moveable platform connected to the base of the support structure, the at least one moveable platform having a top surface and a bottom surface. The apparatus further includes a drive system configured to control the movement of the at least one moveable platform. The drive system includes an actuator secured to the at least one moveable platform. A set of rollers is secured to the base and in contact with the at least one moveable platform. The set of rollers is configured to restrict movement of the at least one moveable platform to a one-dimensional axis. The apparatus further includes a first sensor located above the support structure. The first sensor is configured to determine an amount of downward pressure exerted on the training apparatus by a user. A second sensor is located above the support structure which is configured to determine the position of the user on the at least one moveable platform. The apparatus also includes a control system having a programmable logic controller (PLC). The PLC is configured to control the drive system and to receive data from the first and second sensors. In response to data received from the first and second sensors, the actuator changes the movement of the moveable platform.

### CROSS REFERENCE TO RELATED APPLICATIONS

The present application is a continuation of U.S. patent application Ser. No. 15/017,911, filed Feb. 8, 2016, which claims priority to U.S. provisional patent application No. 62/113,817 filed Feb. 9, 2015. The entire disclosure contents of these applications are herewith incorporated by reference into the present application.

### BACKGROUND

Anyone can fall on a slippery surface. The elderly are at particularly increased risk of falls with increasing age, as demonstrated by staggering fall statistics that rise significantly with each decade of life above age sixty. This is a result of a natural slowing of reflexes as well as deconditioning and debility.

Falls are the leading cause of death by injuries among those aged 65 and over. Each year, more than 700,000 people suffer injuries from falls that result in hospitalizations. As people age, they are increasingly susceptible to falls as a consequence of diminished strength and delayed reaction time.

Falls are associated with increased length of stay, higher rates of discharge to nursing homes, and greater health care utilization. Falls cause significant harm to patients. 30-51% of falls in hospitals result in some injury, varying from bruises to severe wounds or bone fractures.

Falls among the elderly commonly lead to a loss of independence, particularly with activities of daily living (ADLs), reducing an individual's sense of dignity. Unfortunately, falls are the top reason individuals get admitted to nursing homes. The aging baby boomer population will further increase the demand for new technologies that keep them from falling and allow them to maintain an active lifestyle.

It has been shown that falls among the elderly have been reduced after a short training session on a device that simulates trips and slips. Such a device has the potential to vastly improve the unacceptably high morbidity and mortality from fall injuries, and also improve quality of life for patients while reducing the overall cost of healthcare. Thus, a need exists for such a training apparatus that is both practical to use in a clinical setting, and effective in simulating slips in a controlled and safe environment. With strength training and reflex training, users should achieve a reduced likelihood of falling for a long period of time after each training session.

### SUMMARY

A gait training apparatus is disclosed that reduces physical harm to subjects at risk of suffering fall injuries. The apparatus can be used during physical therapy, by companies seeking to reduce on the job injuries, and during general fitness training to improve users' reflexes, balance, and proprioception.

In one embodiment, the gait training apparatus includes a support structure having a base, at least one moveable platform connected to the base of the support structure, the at least one moveable platform having a top surface and a bottom surface. The apparatus further includes a drive system configured to control the movement of the at least one moveable platform, the drive system including an actuator secured to the at least one moveable platform. A set of rollers is secured to the base and in contact with the at least one moveable platform, the set of rollers being configured to restrict movement of the at least one moveable platform to a one-dimensional axis. The apparatus further includes a first sensor located above the support structure, the first sensor being configured to determine an amount of downward pressure exerted on the training apparatus by a user, a second sensor located above the support structure, the second sensor being configured to determine the position of the user on the at least one moveable platform, and a control system including a programmable logic controller (PLC), the PLC being configured to control the drive system and to receive data from the first and second sensors. In response to data received from the first and second sensors, the actuator changes the movement of the moveable platform.

A method of training a user to prevent injury is also provided. The method includes providing an apparatus comprising a moveable platform that a user traverses at a walking speed, the apparatus further including a control system having a programmable logic controller (PLC). The PLC initiates an assessment phase to determine a risk factor for the user and performs a simulation phase. The simulation phase includes varying movement of the moveable platform based on output received from a plurality of sensors associated with the apparatus and varying the frequency of actuation of the moveable platform based on the risk factor determined during the assessment phase. The PLC further records data of the user during the simulation phase and analyzes the recorded data and provides feedback.

A non-transitory computer-readable medium is also disclosed. The non-transitory computer-readable medium stores instructions that, when executed by a computing device, cause the computing device to initiating an assessment phase to determine a risk factor for the user, perform a simulation phase, the simulation phase including varying movement of a moveable platform based on output received from a plurality of sensors associated with an apparatus, and varying the frequency of actuation of the moveable platform based on the risk factor determined during assessment phase. The computing device is further caused to record data of the user during the simulation phase and analyze the recorded data and provide feedback.

### BRIEF DESCRIPTION OF THE DRAWINGS

FIG. 1 shows one embodiment of a gait training apparatus in accordance with the present application having a movable platform;

FIG. 2 shows another embodiment of the gait training apparatus in accordance with the present application;

FIG. 3 is a close-up view of the movable platform of the gait training apparatus;

FIG. 4 shows details of a drive system attached to the movable platform;

FIG. 5 shows yet another embodiment of the gait training apparatus having two movable platforms;

FIG. 6 shows yet another embodiment of the gait training apparatus in which the platform displaces along two axes which are perpendicular to one another;

FIG. 7 shows yet another embodiment of the gait training apparatus;

FIG. 8 is a flow chart showing example operation of the gait training apparatus in an automated mode;

FIG. 9 is a force gauge chart indicating no fall event;

FIG. 10 is a displacement chart demonstrating the displacement of the trolley over time;

FIG. 11 is a force gauge chart during a supported fall event;

FIG. 12 is a force gauge chart during an unsupported fall event;

FIG. 13 is a gait chart demonstrating the trolley's position over time and velocity during a fall event;

FIG. 14 is a chart showing example displacement data of the platform itself during a perturbation; and

FIG. 15 is a flow chart showing example operation of the gait training apparatus in a manual mode.

### DETAILED DESCRIPTION

A gait training apparatus is disclosed that reduces physical harm to users by preventing or limiting injuries from falling and improving overall mobility and reaction time by facilitating neuromuscular training. Users have been shown to retain certain skills and reflexes after repeated exposures to simulated slips and trips. The apparatus exposes users to slips and trips in a controlled setting to simulate falls that commonly occur in peoples' normal environments (e.g., slippery surfaces indoors and icy surfaces outdoors).

In one embodiment, the apparatus includes a moveable platform that a subject traverses at walking speed, the platform having a changeable surface in order to vary the friction. The apparatus also includes a drive system for actuating the platform. Perturbations or movements of the platform are carried out with an actuator that can be manually controlled or automatically controlled by a programmable logic controller (PLC). In either control system, the speed of the actuation, the acceleration of the actuation, and the amplitude of the actuation are fixed during each session. The PLC is programmed with algorithms having specific sequences of actuations that will result in perturbations of the platform on which the user walks back and forth. The PLC also factors in the subject's direction of travel. The apparatus further includes sensors that determine the position of the user along the platform. The PLC can compile positional data and timer inputs to derive the velocity of the subject. The PLC can also record the downward force a subject places upon a safety harness during slip and trip events using a load sensor. A slip occurs when a user's center of mass shifts posteriorly leading the subject to land on his/her backside. A trip is the opposite type of fall in which the user's center of mass shifts anteriorly, thereby causing the subject to land on his/her front-side and often causing a fall on outstretched hand (FOOSH) injury. Further, the apparatus includes a base that allows the platform to move at low friction along one or two axes of travel and houses the drive system, and having a central fulcrum that raises sides to simulate uphill, downhill and traverse hill conditions. The apparatus is connected to the Internet through a managed switch to provide an enterprise system with the results of a user's treatment session, which can be entered into a medical chart and/or shared with a referring physician.

Point of care decision-making is the end goal of the apparatus. With the added information, the physician can make informed decisions on the user's clinical condition such as activities of daily living, level of independence, subsequent fall risk, and of gait stability.

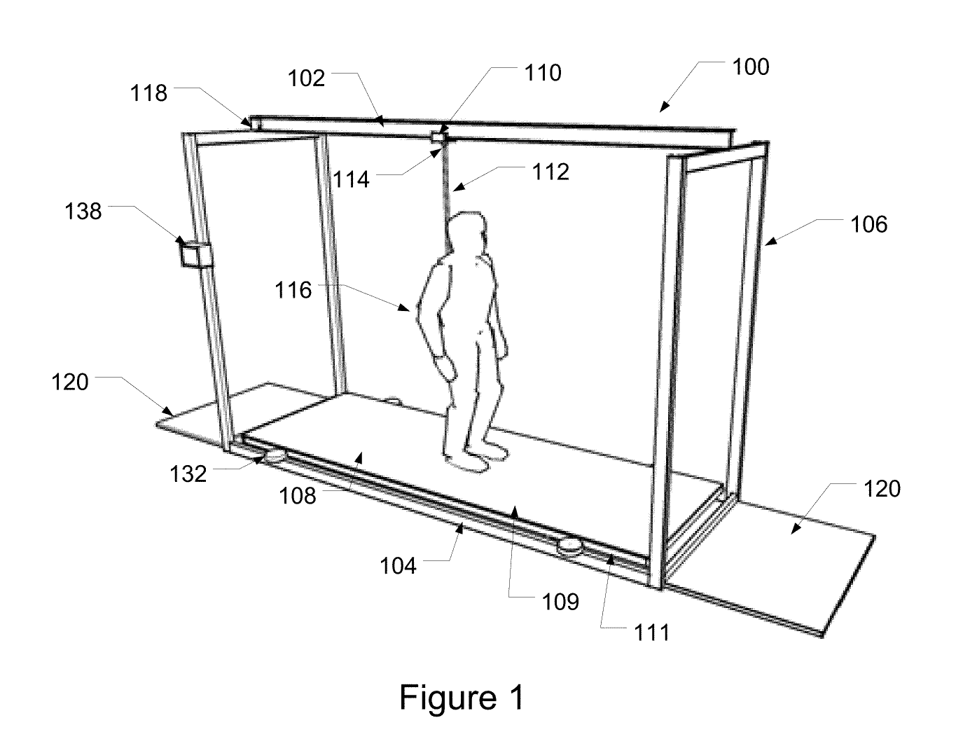

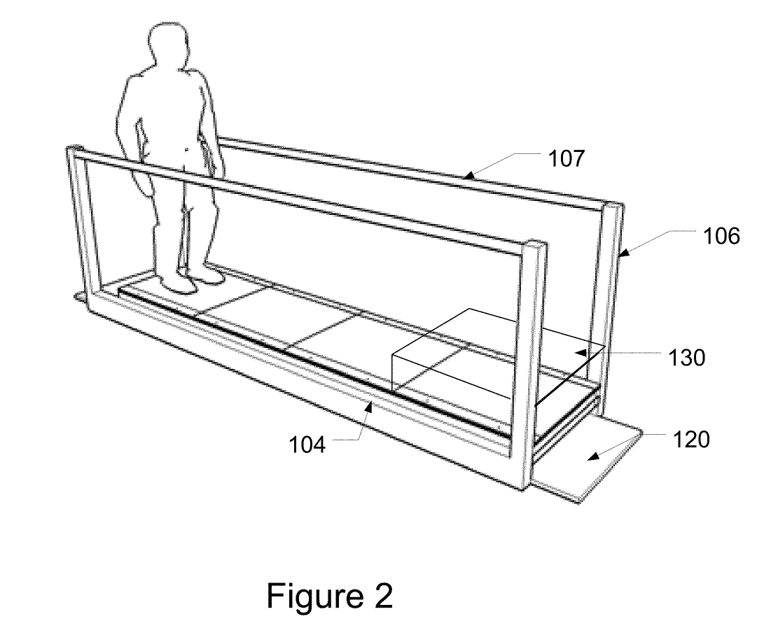

FIG. 1 shows a first embodiment of a gait training apparatus 100 where a moveable platform 108 displaces front and back along one axis. The apparatus includes a mechanical structure defined as a user support structure or scaffold and a movable platform 108. The support structure may include an I-beam 102, a base 104, and one or more vertical columns 106. The user support structure can have four—or more or less—vertical columns 106 connected horizontally at their base and their peak. The beam 102 is located along the top of the support structure and runs the along a parallel centerline to the length of the base 104. A trolley is connected to the bottom of the I-beam 102. The trolley 110 is configured to move along the length of the I-beam 102 utilizing rollers, friction reducing parts, or other bearings that move along the I-beam's length. The trolley 110 is linked to a rope or strap 112 that is adjustable in length and connected to a safety harness (not shown), such as a vest or a belt. A load cell sensor 114 is connected either in series or in parallel between the trolley 110 and the safety harness. In an alternate embodiment, an extruded upside down U-shaped profile may be used instead of the I-beam 102 with a trolley 110 that runs inside of the extrusion's center cavity. In yet another embodiment, the user support structure can be partially external to the apparatus (for example, a harness, trolley, and rail system fixed to a pre-existing structure, such as the ceiling of a room). In this embodiment, the user support structure includes a base 104 and one or more vertical columns 106, as shown in FIG. 2. Further, one or more barriers 107 are provided to prevent patients from walking off the sides of the moveable platform 108. Other configurations are also possible.

The load cell 114 is used to determine the amount of downward pressure exerted on the safety apparatus, specifically on the safety harness or the I-beam 102, by a user 116. This information determines when falls or losses of balance occur and measures their magnitudes.

A laser displacement sensor (or alternate type of sensor) 118 is positioned on one end of the support structure's overhead I-Beam 102. The laser of the sensor tracks the moving trolley 110 to determine its position, and hence the position of the user 116. The position data and its change over time indicate the user's speed and direction of motion. It should be understood that the sensor 118 may be positioned in other locations on the apparatus 100, and that additional displacement sensors may also be positioned on the apparatus 100.

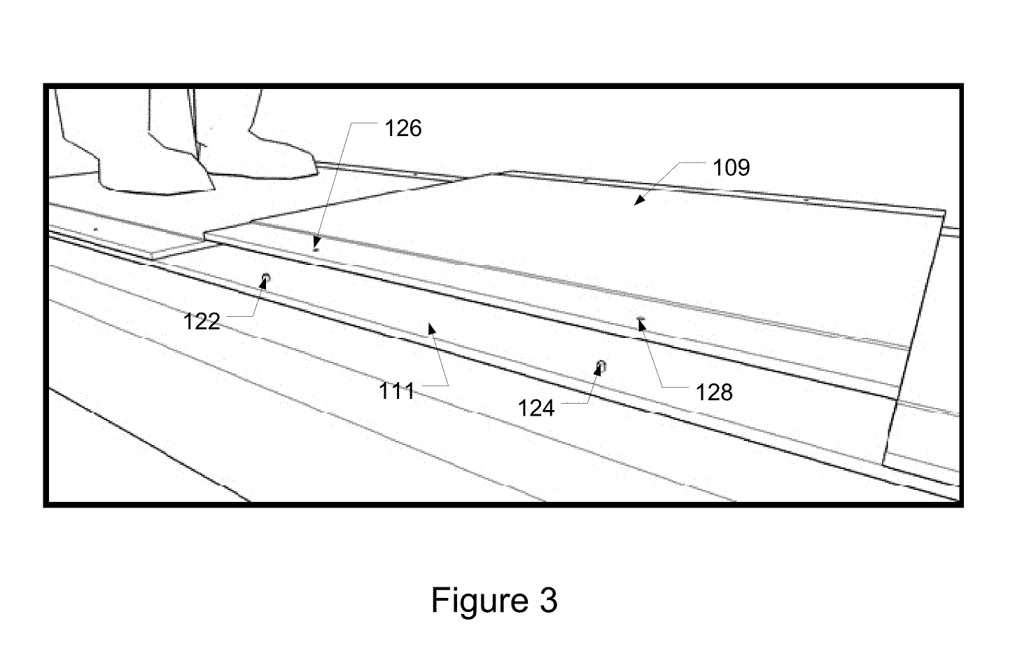

The base 104 may include a rectangular structure upon which the movable platform 108 rests. The base 104 may also include safety pads 120 at both ends of its length to prevent actuation while an object is in its path. The movable platform 108 can be traversed by the user 116 by walking several steps in either direction along its length. The movable platform 108 may include a top surface 109 attached to a bottom surface 111. The top surface 109 of the movable platform 108 may comprise a variable material, and the bottom surface 111 may be a rigid plate. The top surface 109 may include positioning holes 122 and 124 that are fixed onto the bottom surface 111 by side support pins 126 and 128, as shown in FIG. 3. Thus, the variable material used on the top surface 109 of the moveable platform 108 can be quickly and easily switched out and changed by lifting the top surface 109 upwards to remove, and laying new material down with different amounts of friction, softness, or various obstacles. The top surface 109 is fixed in place by aligning the positioning holes 122, 124 and support pins 126, 128.

Example variable materials may include polymers of varying friction coefficients, polymers with varying elastomeric properties, and materials of variable density and surface flatness including, for example, Teflon®, nylon, rubber, artificial turf and polyurethane foam. The rigid plates with variable material can also be used to mount obstacles 130 (shown in FIG. 2) that require users to lift their feet while walking.

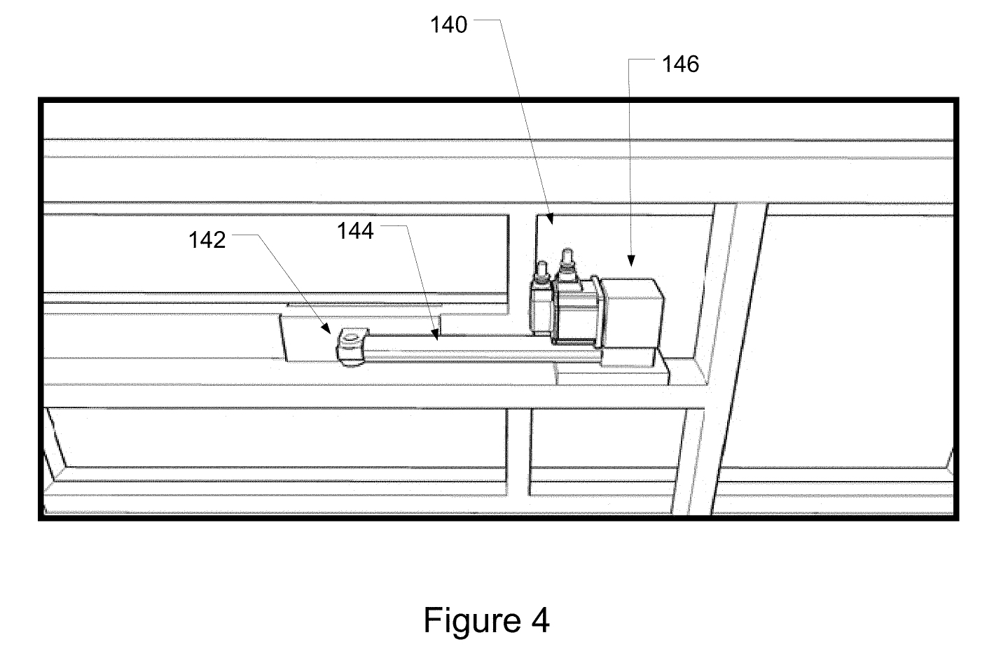

Perturbations, movements and rotations of the movable platform 108 are carried out with an actuator(s) 140 controlled by a programmable logic controller (PLC), which is described in more detail below. The movable platform 108 can be moved abruptly precisely because it is attached to a hinge 142 that is connected to actuator 140 on its underside and rests on rollers, which maintain its vertical position while also creating a low drag condition to the platform's motion along the axis of actuation. The actuator 140 may be a hydraulic powered cylinder connected to compressed air or an electric motor, for example. The actuator 140 controls the position of the apparatus, the direction of motion, and the applied force(s). The actuator 140 is located underneath the movable platform 108, as shown in FIG. 4. In this example, the actuator 140 comprises an electric motor attached to a gear box 146 that rotates a screw 144. The screw then translates, extends, and retracts, which provides a linear motion. The PLC outputs a signal to the electrical motor to indicate direction, speed and position.

In another embodiment, the actuator 140 may be a pneumatic cylinder that relies on an air compressor for high pressure. A valve controller is used to direct compressed air to elongate or reduce the pneumatic cylinder. The controller is connected to an output of the PLC so the algorithm can direct the motion of the cylinder. It should be understood that other types of actuators may be used as well.

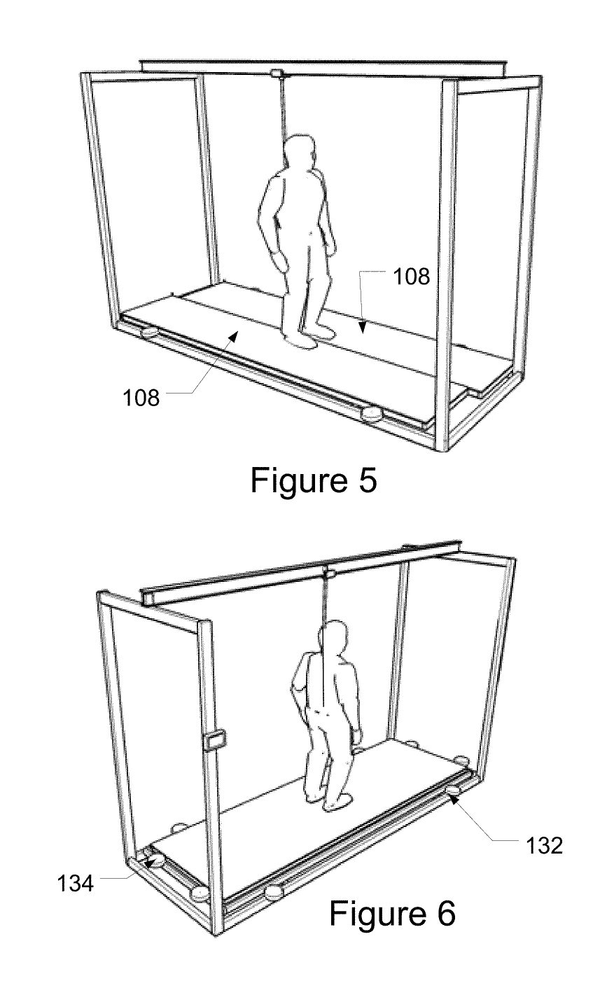

In an alternate embodiment, shown in FIG. 5, the movable platform is split into two separately controlled moving platforms 108, in order to create other types of perturbations to a user's gait. Each platform is connected to a different actuator 140, which enables perturbations to be triggered that can displace one of the user's feet individually, both in tandem, or any combination thereof.

The moveable platform's movement is restricted within a one-dimensional axis by side rollers 132. In another embodiment, shown in FIG. 6, the movable platform can also be moved along a two-dimensional axis. Thus, the movable platform 108 has two axes of motion that are perpendicular to one another and made possible by two sets of rollers 132 and 134. Each set of rollers is set on a different horizontal plane, enabling multiple types of perturbations to the movable platform.

In another embodiment (not shown) the movable platform 108 can be rotated along two axes to represent a tilted environment, both up and down hill as well as traverse (like walking along the side of a hill). The apparatus may include a rotating mechanism, such as a cam using an eccentric disc, causing the simulations to occur at angles similar to conditions that would occur on a hill or other variable surface. Yet another embodiment would rotate the moving platform slightly about the axis of motion for a lopsided walking surface.

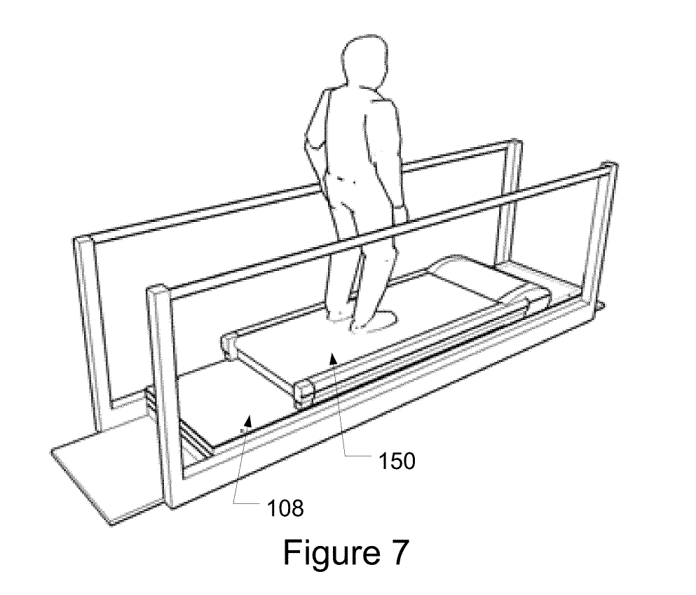

In another embodiment, as shown in FIG. 7, a treadmill 150 can also be mounted to the movable platform 108. The belt on the treadmill 150 can be comprised of various materials to alter the coefficient of friction at different parts of the same belt while walking atop the treadmill.

Referring back to FIG. 1, a human machine interface (HMI) 138 is located on one or more of the vertical columns 106. In alternate embodiments, the HMI 138 may be located on a different part of the apparatus. The HMI 138 may have wireless connectivity, and may comprise a touch screen that allows an operator to control the machine's state between a non-operational mode and an operational mode, as well as an optional assessment phase and a simulation phase (described below). The HMI 138 indicates the status of the simulation and results to the operator at the completion of the simulation. The HMI 138 also is used to input user data. All input mechanisms are defaulted to comply with clinical guidelines.

A network switch (not shown) is used to receive user information from a referring physician or from a database. The network switch may be in an electrical enclosure along with the power supply and PLC. The enclosure may be mounted to the base 30. The enclosure may have a low profile, which allows it to sit beneath the moveable platform 108. The network switch is also used to output the results from a session and store each users' data for future simulations, allowing the operator to assess each users' progress.

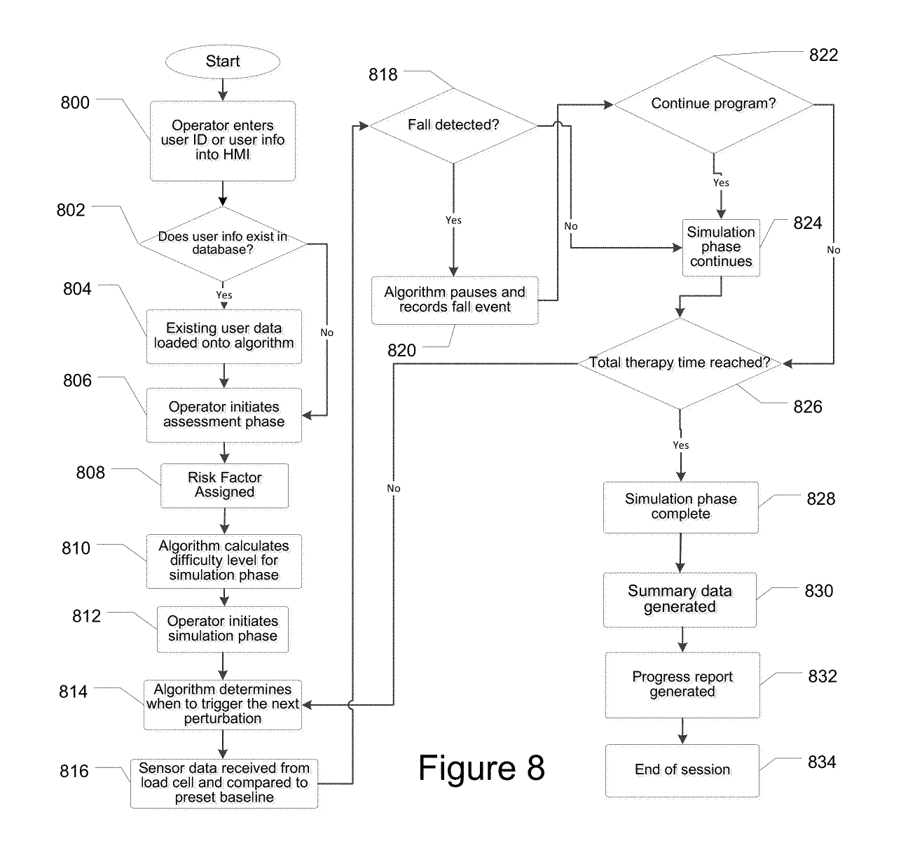

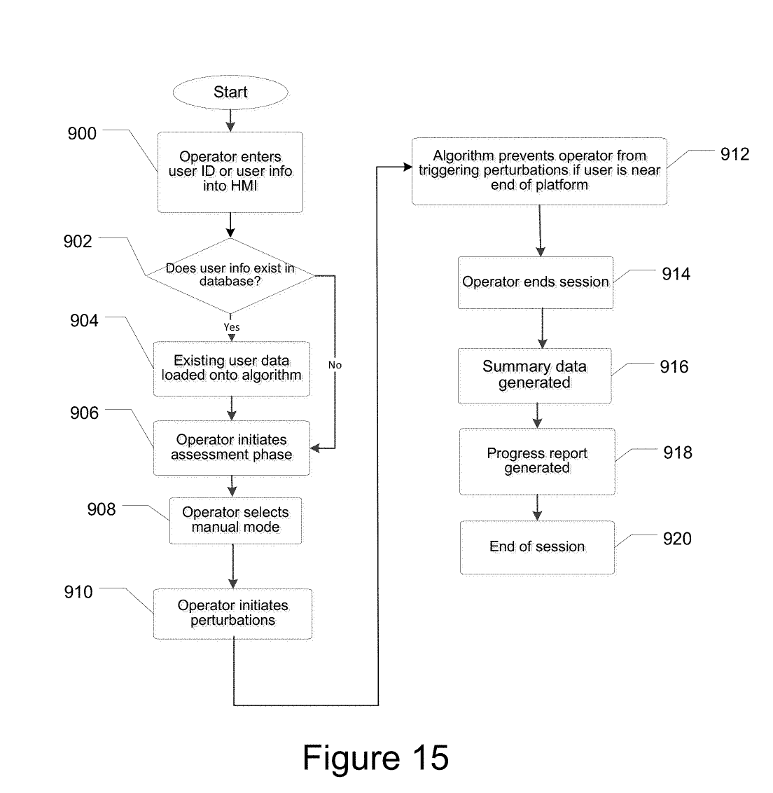

The apparatus further includes a programmable logic controller (PLC), which can operate in an automated mode, shown in FIG. 8, or a manual mode, shown in FIG. 15. The PLC operates on contemporary software utilizing multiple programming logic, one of which includes ladder logic. The difficulty level of the training module may be selected based on the user's previous test results, a referring physician's input, TUG test result, or operator's input (based on other risk factors). The PLC is linked to the load cell 114 and displacement sensors. Outputs from these sensors are factored into algorithms. The PLC outputs information to the apparatus in a way that tailors each training session to each user's abilities. The PLC selects a higher level of difficulty for those users who can tolerate more challenging perturbations. Proficiency is defined as a clinical measure of the user's ability to walk without falling, walk with confidence, achieve improvement in objective measures, or any other clinically relevant metric pertinent to the user.

An algorithm programmed within the PLC recognizes the user's medical history, runs the assessment and simulation phases, analyzes user reaction data, and delivers results. User history is entered at start of each user's session or the process is automated by pulling the information from an enterprise system, such as an Electronic Medical Record (EMR) system. The PLC can adjust the frequency, amplitude and force of actuations based on user factors to select an appropriate level of difficulty. Several gait characteristics of the user are measured and recorded.

In evaluating a user's clinical condition, the apparatus 100 makes note of a number of clinical factors including, but not limited to past medical history, medications, allergies, mental cognitive factors such as dementia, and gait status. Settings of the apparatus may be defaulted to reflect national standards or may be set per the specific clinical caretaker's preferences given appropriately documented clinical need for such a change, learning, clinical risk, or predicted risk. Data inputs include motion detected as a fall or as a compensatory movement. Software may analyze the trend of these movements in order to assess the likelihood that a motion, movement, reaction, compensation, or any combination thereof signifies a clinically relevant process such as, but not limited to, a fall.

The software may also apply the same or different heuristics for the absolute or relative movements signaled by the user, including correlations with prior falls in current or previous session, as well as second order and higher rate functions, and trends that analyze the relationship between movements and fall rates and potential likelihood models, even though these analyses and trends may not be relevant to the clinical diagnosis of abnormal conditions.

A slip is defined as when the movable platform 108 is actuated in a way that a loss of traction is induced between the platform and the users' socks, shoes, or other covering of the feet. The amplitude and speed of the platform's movement required to induce a slip will vary based on the friction coefficients between the two surfaces and certain variables of the user. A trip is defined as when the movable platform 108 is actuated in the opposite direction of the user's walking direction. There may or may not be a loss of traction during this type of perturbation. The amplitude of the board movement can be adjusted and may increase through the duration of the session.

The frequency and amplitudes, which can be defined as a wave function, of platform movements for the induced slips and trips will vary by the software algorithm that factors variables detected by the sensors such as the user's step distance, walking speed, and fall frequency, among other things. If the user is doing well, he/she may be exposed to larger perturbations more frequently and achieve greater improvements to his/her mobility and strength.

In one operational mode, the apparatus 100 is set to an automated operational mode, as shown in FIG. 8. Upon starting, an operator of the apparatus 100 enters a user ID or user information into the HMI 138 at block 800. The PLC then checks to determine if the user ID or information exists in the database of information stored on a server at block 802. If so, then the existing user data is loaded onto an algorithm at block 804 and the operator proceeds to initiate an assessment phase at block 806. If the user ID or information does not exist in the database, then the operator simply proceeds to initiate the assessment phase.

An algorithm programmed into the PLC guides the operator of the apparatus 100 to perform the assessment phase. In another embodiment, the assessment phase is optional and the apparatus can be run in a manual operational mode, which is described below with respect to FIG. 15.

The assessment phase measures the following sequence of events: the amount of time it takes the user 116 to stand up from a seated position, walk ten feet across the platform, turn around, walk back to the starting point, and sit down again (which is known as the Timed Up and Go test (TUG)). This sequence of events allows for a simple assessment of the user's capabilities, and to familiarize the user with the apparatus. A timer is initiated upon completion of this series of movements. The laser displacement sensor 118 signals that the user has reached the length of the moveable platform 108. The user turns around and walks back to the starting position. The laser displacement sensor 118 then signals that user has returned to the starting position, causing the timer to end. The total assessment time is calculated.

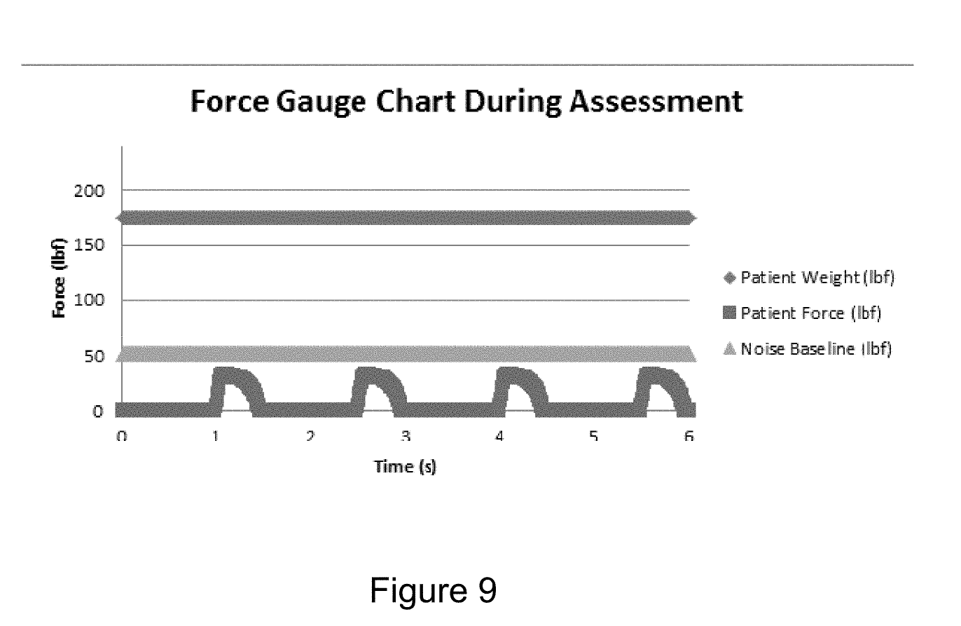

FIG. 9 shows an example force gauge chart during the assessment phase. The chart visually displays data collected by the load cell 114 over a period of 6 seconds that does not result in a fall event. The user has a weight of 175 lbs., which the algorithm assigns a baseline noise weight of 50 lbs. The user takes 4 steps during the 6 seconds displayed, which results in 4 peaks in the user force. Since baseline was never exceeded, no fall event would be recorded by the sensory equipment.

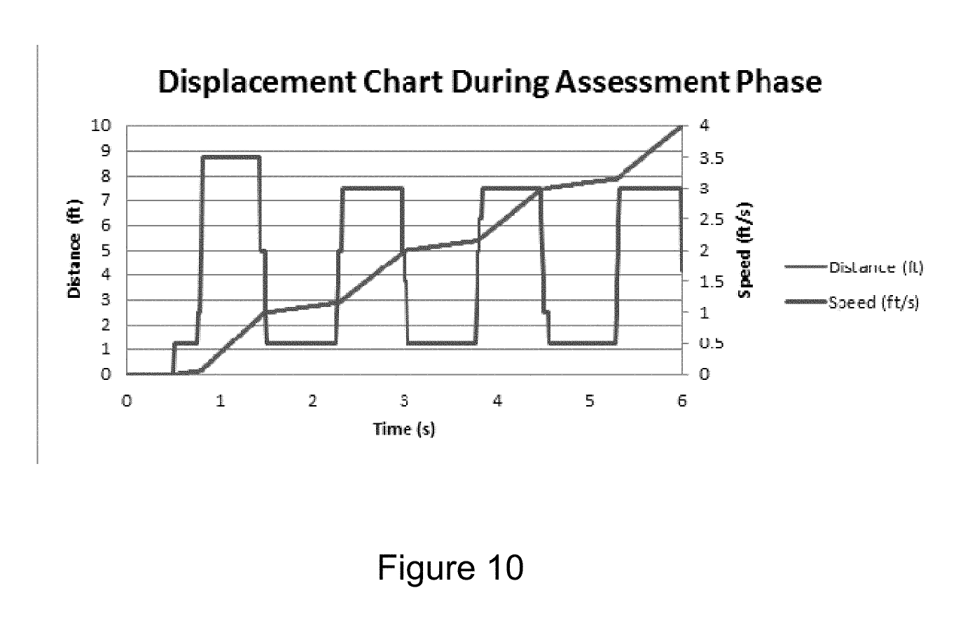

FIG. 10 shows an example displacement chart during the assessment phase. The chart visually displays data collected by the laser displacement sensor 118 over the same period of 6 seconds as the Force Gauge Chart of FIG. 9. The gauge measures the distance (1st vertical axis), while the algorithm interprets the change of distance over time to assign user speed (2nd vertical axis), which peaks periodically as the user walks, since the trolley 110 is not always the same distance from the user. The algorithm also calculates the gait length (ft.) and gait period (s), which are used during the assessment phase to setup the conditions for the simulation phase.

At block 808, the algorithm compares the user's total assessment time with programmed data and prior records, and with referral assessment, then assigns a risk factor to the user. The risk factor (RF) is on a scale of 1 to 4 where:
RF=1 if TUG is 10 seconds or less
RF=2 if: 10<TUG<20 seconds
RF=3 if: 20<TUG<30 seconds
RF=4 if: 30<TUG seconds

At block 810, the assigned risk factor is used by the algorithm to calculate the difficulty level and other user parameters to use during the simulation or treatment phase, such as frequency and number of perturbations. For example, in the equation below at paragraph [0057] the risk factor affects the amount of time between perturbations, and the higher the RF, the longer the mean time between perturbations. Thus, the larger the RF, the lower the difficulty level used during the simulation phase.

After the assessment phase, the operator initiates the simulation phase at block 812 using the HMI 138. During the simulation phase, the movable platform 108 will move according to the PLC's output. At block 814, the algorithm determines when to trigger the perturbations. In one example, the algorithm uses the product of the assigned frequency and a random number generator to assign the time until the next perturbation:

HZ=Rand*(3 seconds*RF)

where HZ=Period between actuations, Rand=Random number between 1 and 3, and RF=Risk Factor (scale of 1 to 4).

The algorithm uses the change in the laser displacement values to determine the direction of the user's travel. The algorithm uses the proximity gauges or an encoder to determine the position of the moving platform. The laser displacement gauge alerts the algorithm when user is near the end of the platform to stall the next perturbation and to prevent injury while turning.

The algorithm factors in timer data, user's direction of travel, and position of the moveable platform to determine when to trigger the next perturbation.

A perturbation event is recorded and the algorithm receives sensor data from load cell 114, and compares it to a baseline (which is a level of acceptable load on the load cell) at block 816. If the load cell 114 detects a fall at block 818 (i.e., if a force greater than a preset fraction of the user's weight is detected), then the algorithm pauses the simulation phase for a preset time and records fall event at block 820. The HMI screen then notifies the operator of the fall event and the operator selects whether to continue or abort the program at block 822. If no fall is detected, then the simulation phase continues at block 824.

While the user moves, there are information points monitoring the user's behavior and reaction. When the movable platform 108 moves due to an inciting factor or a sequence of inciting factors, the user will react accordingly. A series of user reactions, compensations, and other movements are broken down as a function of the inciting event.

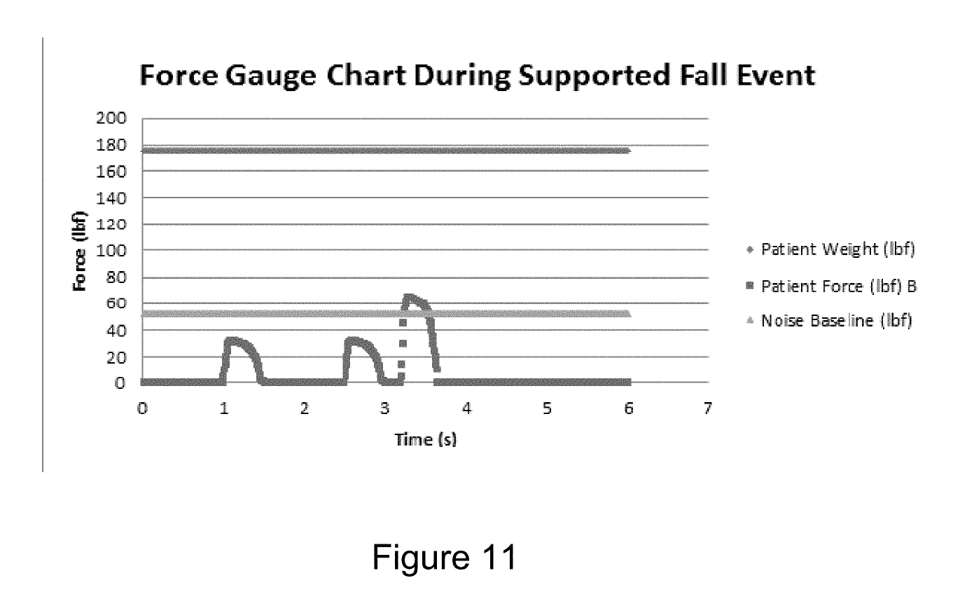

FIG. 11 shows a force gauge chart during a supported fall event. The chart visually displays data collected by the load cell 114 during an 8 second period that includes a supported fall event, which is a partial fall where a portion of the patient's weight is supported by the safety harness. The force on the load cell 114 exceeds the noise baseline but not the user's weight, thereby indicating a supported fall event at just over 3 seconds.

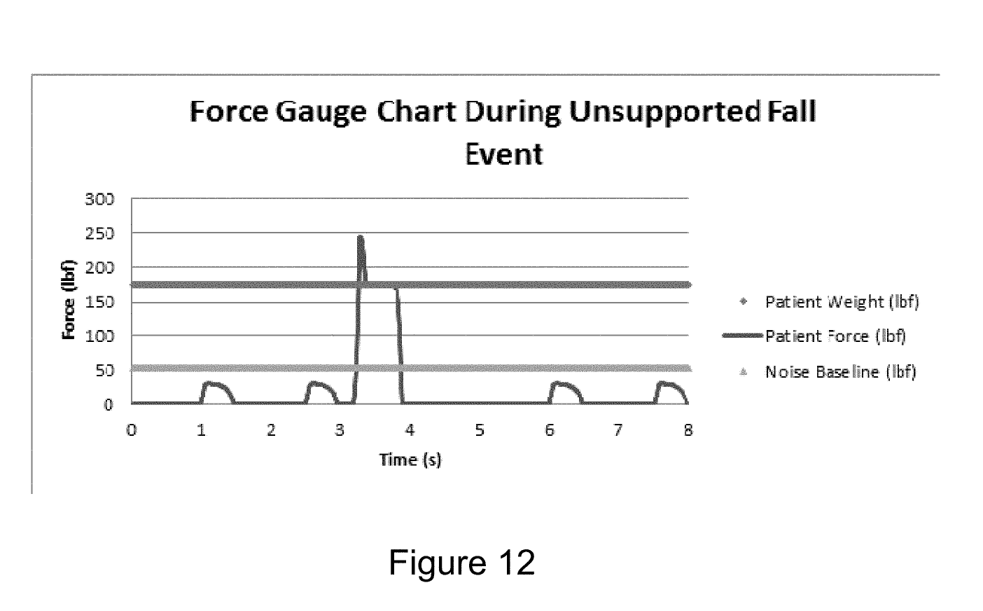

FIG. 12 shows a force gauge chart during an unsupported fall event. The chart visually displays data collected by the load cell 114 during an 8 second period that includes an unsupported fall event, which is a complete fall in which the patient's entire weight is supported by the safety harness and detected by the load cell 114. The force on the load cell exceeds both the noise baseline and the user weight, thereby indicating an unsupported fall event at just over 3 seconds. The user then takes about 2 seconds before returning to the session.

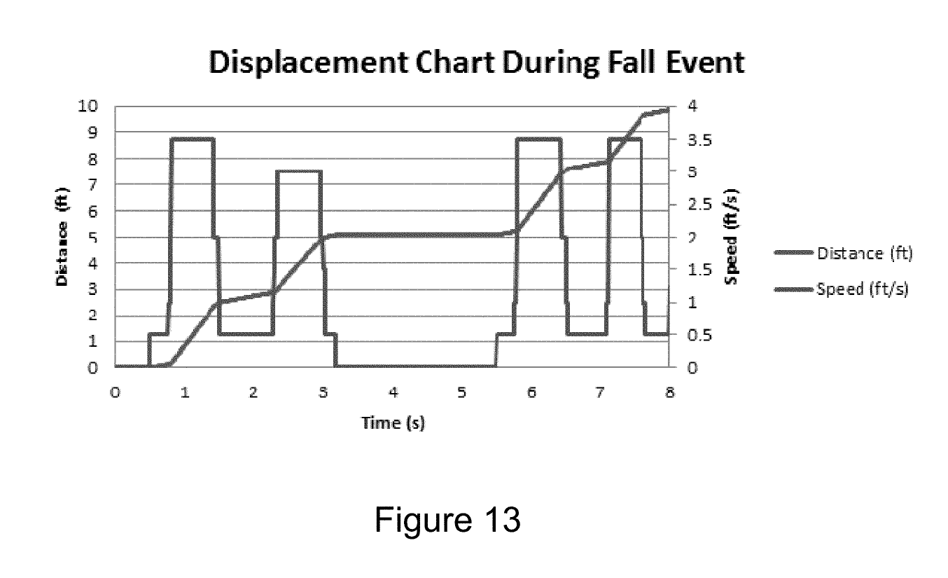

FIG. 13 shows a displacement chart during a fall event. The chart visually displays data collected by the laser displacement sensor 118 over the same period of 8 seconds as FIG. 12. The user does not move immediately following the fall event at over 3 seconds. The user returns to the session at approximately 5.5 seconds.

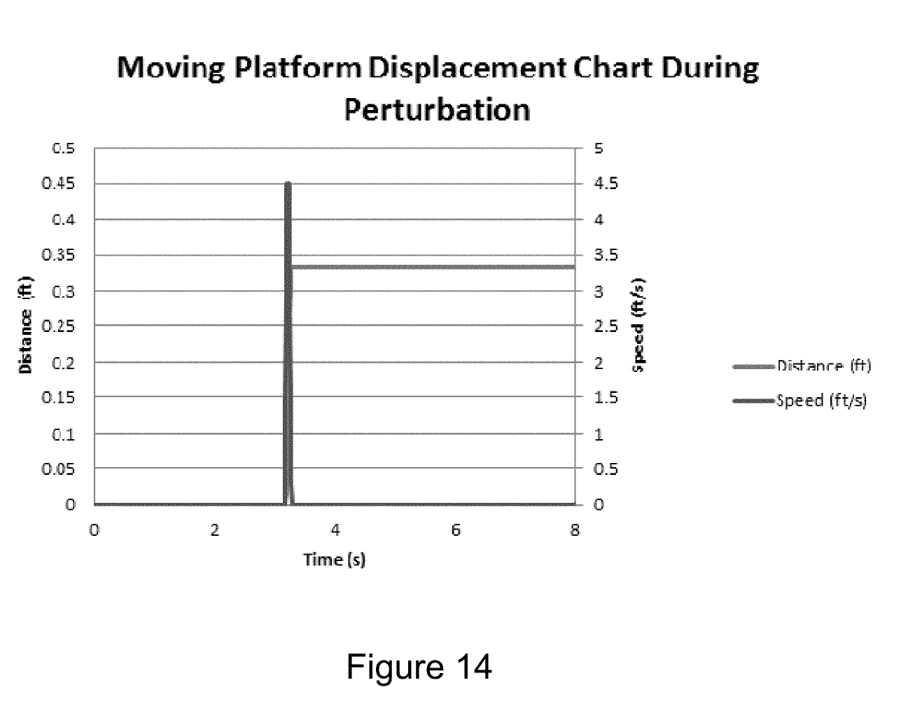

FIG. 14 shows the displacement of the moveable platform during a perturbation. The chart visually displays data collected by the moving platform proximity sensors over the same period of 8 seconds as FIG. 12. The moveable platform is shown to move suddenly at about 3 seconds and stay in that position for the remaining period of time. Assume one single inciting event: the moveable platform moves in a slip. At the first half of the inciting event, the board is increasing in acceleration, at the second half of the inciting event, the board is decreasing in acceleration. The user's reaction, compensation, and other movements during the two phases will be in response to a positive and then negative acceleration. This waveform function of acceleration reflects differing phases of user behavior. The apparatus chronicles all sensor inputs, the user's behavior at each phase of the positive, then negative acceleration.

After a series of detected falls, the algorithm determines if the total therapy time has been reached at block 826. A standard time is about ten (10) minutes, but is adjustable. The time of ten (10) minutes is based on clinical findings on the optimal time to reduce fall injuries by 90%. If the total time has been reached, the simulation phase is complete at block 828. If not, then the algorithm determines when to trigger the next perturbation at block 814.

At the conclusion of the simulation phase, data is tabulated and analyzed to determine progress to reducing falls and increasing mobility of the user. At block 830, the algorithm provides summary data including the number of traverses, number of perturbations, number of fall events, and other data points. A program on a server evaluates the user's dataset, compares to previously collected data and preset baselines, and provides a full analysis of the training session. A progress report is then generated at block 832 which is accessible and stored on an enterprise system at the site of treatment. Copies can be distributed to the referring physician and the user. The training session is then complete at block 834.

The data provided by the algorithm can be integrated into a centralized database where it can be analyzed in real-time along with other vital signs or other clinical data. The data can be displayed as per the perspective of the moveable platform movement, user movement, or a ratio thereof. The data converts the information measured by the disclosed apparatus into point of care information and may influence real-time decision making and prospective clinical decisions. Decisions include whether to provide the user with additional clinical services, limit activities of daily living, restrict activity, initiate or modify clinical care, refine clinical protocol, or other clinically relevant decisions that incorporate the data from the apparatus and user's clinical needs.

Alternatively, in manual mode, shown in FIG. 15, the operator pushes a button to trigger actuations without use of a programmed algorithm. The manual operator must also observe the patient for fall occurrences and keep time of the patient's session. The operator can enter these values into the PLC or keep their own records.

As shown in block 900, an operator of the apparatus 100 enters a user ID or user information into the HMI 138. The PLC then checks to determine if the user ID or information exists in the database of information stored on a server at block 902. If so, then the existing user data is loaded onto an algorithm at block 904 and the operator proceeds to initiate an assessment phase at block 906. If the user ID or information does not exist in the database, then the operator simply proceeds to initiate the assessment phase.

The operator asks the user to walk the length of the moveable platform 108, turn around, and return to the starting position. This motion initiates a timer. The laser displacement sensor 118 signals that the user has reached the length of the moveable platform 108. The user turns around and walks back to the starting position. The laser displacement sensor 118 then signals that user has returned to the starting position, causing the timer to end. The total assessment time is calculated.

The operator then selects manual mode and initiates a gait training session by asking user to start walking back and forth on the moveable platform 108 at block 908. At block 910, the operator initiates intermittent perturbations by pressing a button on the HMI 138. The operator will determine the frequency and timing of perturbations based on their professional training. The laser displacement sensor 118 alerts the algorithm when the user is near the end of the moveable platform 108 and the operator is prevented from triggering perturbations when the user is near the end of the moveable platform 108 at block 912. The operator then ends the session by pressing a button on the HMI 138 at block 914.Data from the session is tabulated and analyzed to determine progress to reducing falls and increasing mobility of the user. At block 916, the algorithm provides summary data including the number of traverses, number of perturbations, number of fall events, and other data points. A program on a server evaluates the user's dataset, compares to previously collected data and preset baselines, and provides a full analysis of the training session. A progress report is then generated at block 918 which is accessible and stored on an enterprise system at the site of treatment. Copies can be distributed to the referring physician and the user. The training session is then complete at block 920.

Other embodiments of motion are based upon the following: tactile based pressure sensors, heat sensors, and reed sensors. In the embodiment with pressure sensors, the moveable platform responds to fluxes in pressure changes induced by the forward gait motion of the user. As the user takes one stride forward, the pressure changes in the platform indicate forward motion and will induce an actuation to move the platform and replicate an extrinsic slip or trip. Alternatively, heat sensors may be used to detect changes in temperature emanating from the user's feet and induce an actuation. Another alternative may be to use reed sensors for this purpose.

In the previous embodiments, a single slip and or trip event is defined. However, in other embodiment, multiple events following one another in a sequential manner may take place. The pattern of multiple slips and trips—referred also as inciting events—can be random, patterned by a fixed rate, patterned by a varying rate, or patterned by a varying rate that itself changes over time. In addition to the pattern, the quantity of slips or trips within one single inciting event can be any number greater than one.

While various aspects and embodiments have been disclosed, other aspects and embodiments will be apparent to those skilled in the art. The various aspects and embodiments provided in this disclosure are for purposes of illustration and are not intended to be limiting, with the true scope being indicated by the following claims, along with the full scope of equivalents to which the claims are entitled.

1. A gait training apparatus comprising:
a support structure having a base;
at least one moveable platform connected to the base of the support structure, the at least one moveable platform comprising a top surface and a bottom surface;
a drive system configured to control movement of the at least one moveable platform, the drive system including an actuator secured to the at least one moveable platform;
a set of rollers secured to the base and in contact with the at least one moveable platform, the set of rollers being configured to restrict movement of the at least one moveable platform to a one-dimensional axis;
a first sensor located above the support structure, the first sensor being configured to determine an amount of downward pressure exerted on the gait training apparatus by a user;
a second sensor located above the support structure, the second sensor being configured to determine position of the user on the at least one moveable platform; and
a control system including a programmable logic controller (PLC), the PLC being configured to control the drive system and to receive data from the first and second sensors;
wherein in response to the data received from the first and second sensors, the actuator changes the movement of the at least one moveable platform.

2. The gait training apparatus of claim 1 wherein the support structure further comprises a top portion and at least one vertical column connecting the base to the top portion.

3. The gait training apparatus of claim 2 further comprising a trolley apparatus secured to the top portion, and a strap extending from the trolley apparatus, the strap being configured to support weight of the user.

4. The gait training apparatus of claim 1 wherein the top surface of the at least one moveable platform comprises a variable material and the bottom surface of the at least one moveable platform comprises a rigid plate.

5. The gait training apparatus of claim 4 wherein the variable material comprises polytetrafluoroethylene, nylon, rubber, artificial turf, or polyurethane foam.

6. The gait training apparatus of claim 1 wherein the top surface of the at least one moveable platform is removable.

7. The gait training apparatus of claim 6 wherein the top surface of the at least one moveable platform is secured to the bottom surface of the at least one moveable platform by at least one fastener.

8. The gait training apparatus of claim 1 further comprising at least two moveable platforms, wherein each moveable platform is connected to a separate actuator.

9. The gait training apparatus of claim 1 wherein the actuator is a motor.

10. The gait training apparatus of claim 1 further including a second set of rollers connected to the base and in contact with the at least one moveable platform to allow movement of the at least one moveable platform along a two-dimensional axis.

11. The gait training apparatus of claim 1 further comprising a rotating mechanism configured to tilt the at least one moveable platform.

12. The gait training apparatus of claim 1 further comprising a network switch configured to connect to the Internet.

13. The gait training apparatus of claim 1 further comprising a human machine interface (HMI) associated with the apparatus configured to allow an operator to control apparatus.

14. The gait training apparatus of claim 1 further comprising a treadmill mounted to the at least one moveable platform.

15. A method of training a user to prevent injury, the method comprising:
providing an apparatus comprising a moveable platform that a user traverses at a walking speed, the apparatus further comprising a control system having a programmable logic controller (PLC), the PLC performing the following steps:
initiating an assessment phase to determine a risk factor for the user;
performing a simulation phase, the simulation phase comprising:
varying movement of the moveable platform based on output received from a plurality of sensors associated with the apparatus; and
varying a frequency of actuation of the moveable platform based on the risk factor determined during the assessment phase;
recording data of the user during the simulation phase; and
analyzing the recorded data and providing feedback.

16. The method of claim 15 wherein the risk factors include the user's medical history, weight, age, and gait characteristics.

17. The method of claim 15 wherein the data includes gait characteristics and fall occurrences of the user.

18. The method of claim 15 further comprising displaying the feedback to the user.

19. The method of claim 15 further comprising transmitting the feedback via a network connection.

20. A non-transitory computer-readable medium storing instructions that, when executed by a computing device, cause the computing device to perform functions comprising:
initiating an assessment phase to determine a risk factor for the user parameters;
performing a simulation phase, the simulation phase comprising:
varying movement of a moveable platform based on output received from a plurality of sensors associated with an apparatus; and
varying a frequency of actuation of the moveable platform based on the risk factor determined during the assessment phase;
recording data of the user during the simulation phase; and
analyzing the recorded data and providing feedback.
